(19) United States
(12) Patent Application Publication
(10) Pub. No.: US 2002/0143374 A1
(43) Pub. Date: Oct. 3, 2002

(54) Thermal acupuncture and moxibustion device

(76) Inventor: Chih-Ho Tai, Taipei (TW)

Correspondence Address: VARNUM, RIDDERING, SCHMIDT & HOWLETT LLP, P.O. BOX 352, GRAND RAPIDS, MI, 49501, US

(21) Appl. No.: 09/825,387

(22) Filed: Apr. 3, 2001

(51) Int. Cl. A61F 7/00; A61F 7/12

(52) U.S. Cl. 607/96

(57) ABSTRACT

A thermal acupuncture and moxibustion device is disclosed. A dosage detection unit decodes and analyzes signals from a temperature sensor. A buffering amplifier amplifies signals from the dosage detection unit. An analog/digital converting unit converts the amplified signals into digital signals. A microcomputer operation unit determines a desired dosage from the digital signals based on a dosage control software, and sends out pulse signals representing the desired dosage. A thermal acupuncture and moxibustion digital/analog signal amplifier converts the pulse signals into analog signals and amplifies the same. A thermal acupuncture and moxibustion driving unit drives a heating wire so as to adjust the temperature of the thermal acupuncture and moxibustion unit.

### BACKGROUND OF THE INVENTION

1. Field of the Invention

The present invention relates to a thermal acupuncture and moxibustion device which has an acupuncture and moxibustion unit, and a temperature control circuit for supplying electrical power to the acupuncture and moxibustion unit to increase the temperature, so as to treat the human body with a thermal acupuncture and moxibustion device.

2. Description of Related Art

In the acupuncture and moxibustion therapy of the traditional Chinese medical skill, it is known to use acupuncture needles with burning moxa or to directly use burning moxa cones to press on the acupuncture points of the human body, thereby treating the human body. However, such a thermal acupuncture and moxibustion therapy may have the side effects of causing a burn injury and producing a scar. From the viewpoint of modern science, there are no constant scientific parameters provided for controlling the strength, duration, and temperature of the acupuncture stimulation. Nevertheless, based on the historical experiences of ancient China, the thermal acupuncture and moxibustion therapy does provide a satisfactory curative effect, and has a low medical treatment cost due to its widespread use. Therefore, it is desired to provide a correct and constant mode to perform the thermal acupuncture and moxibustion therapy for conveniently achieving the curative effect.

### SUMMARY OF THE INVENTION

Accordingly, the object of the present invention is to provide a thermal acupuncture and moxibustion device capable of controlling the strength, duration, and temperature of the acupuncture stimulation, so as to achieve an excellent curative effect in a convenient manner.

To achieve the object, the thermal acupuncture and moxibustion device in accordance with the present invention has a thermal acupuncture and moxibustion unit and a temperature control circuit. The temperature control circuit comprises: a dosage detection unit connected a temperature sensor for decoding and analyzing signals from the temperature sensor; a buffering amplifier for amplifying signals from the dosage detection unit; an analog/digital converting unit for converting the amplified signals into digital signals; a microcomputer operation unit for determining a desired dosage from the digital signals based on a dosage control software, and sending out pulse signals representing the desired dosage; a thermal acupuncture and moxibustion digital/analog signal amplifier for converting the pulse signals into analog signals and amplifying the same; and a thermal acupuncture and moxibustion driving unit connected to an output of the thermal acupuncture and moxibustion digital/analog signal amplifier for driving a heating wire so as to adjust the temperature of the thermal acupuncture and moxibustion unit.

Other objects, advantages, and novel features of the invention will become more apparent from the following detailed description when taken in conjunction with the accompanying drawings.

### BRIEF DESCRIPTION OF THE DRAWINGS

FIGS. 1A, 1B and 1C are the circuit diagram of a thermal acupuncture and moxibustion temperature control circuit in accordance with the present invention;

FIG. 2 is the top cross sectional view of a thermal acupuncture and moxibustion unit in accordance with the present invention;

FIG. 3 is the side cross sectional view of the thermal acupuncture and moxibustion unit in accordance with the present invention;

FIG. 4 schematically illustrates a practical application of the thermal acupuncture and moxibustion device in accordance with the present invention; and

FIG. 5 shows the operational flow of the thermal acupuncture and moxibustion device in accordance with the present invention.

### DETAILED DESCRIPTION OF THE PREFERRED EMBODIMENT

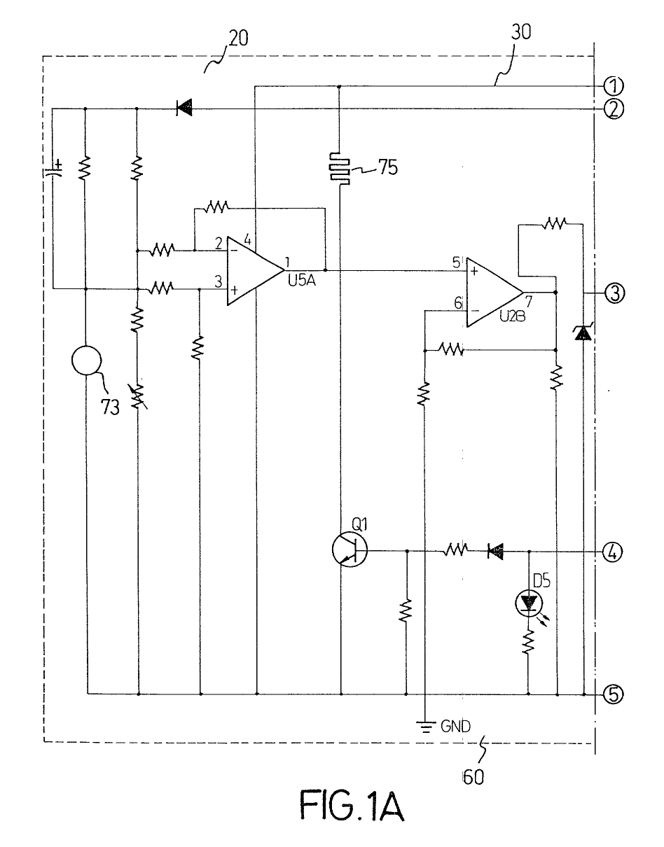

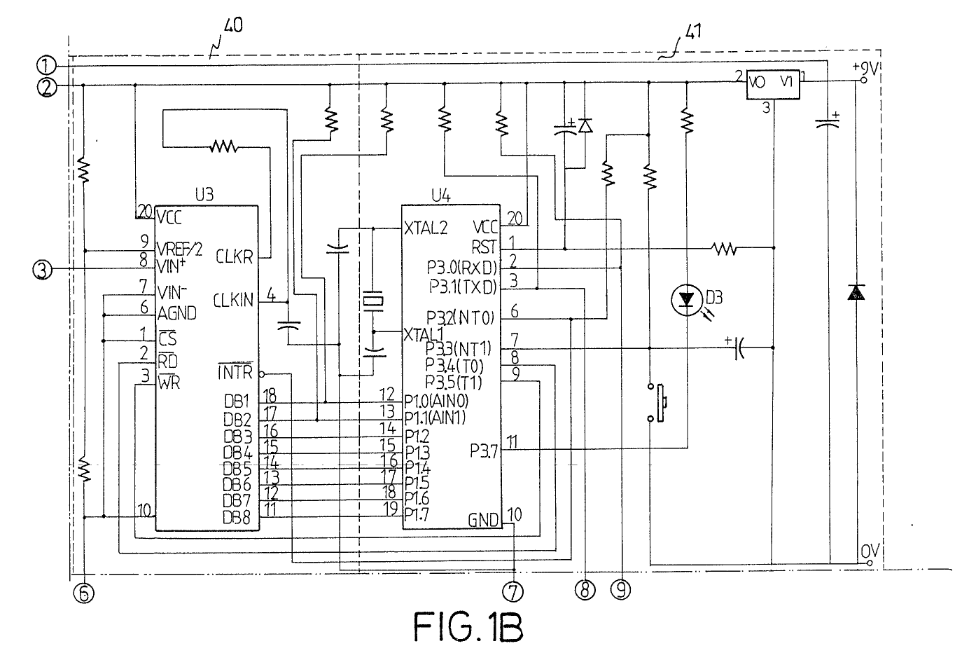

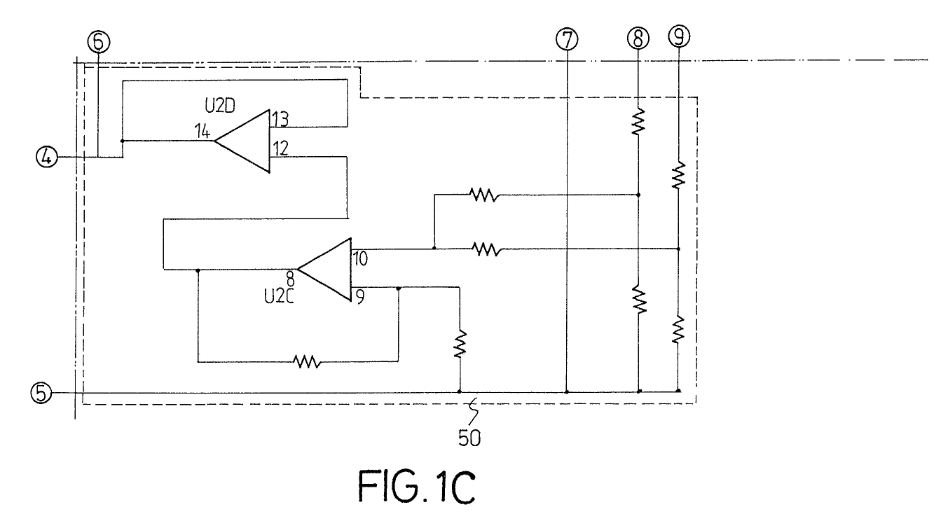

FIGS. 1A, 1B and 1C depict the circuit diagram of a thermal acupuncture and moxibustion temperature control circuit in accordance with the thermal acupuncture and moxibustion device of the present invention, which includes a dosage detection unit 20, a buffering amplifier 30, an analog/digital converting unit 40, a microcomputer operation unit 41, a thermal acupuncture and moxibustion digital/analog signal amplifier 50, and a thermal acupuncture and moxibustion driving unit 60.

The dosage detection unit 20 is connected to a temperature sensor 73 for decoding and determining the signal from the temperature sensor 73. The buffering amplifier 30 is provided to amplify the determined signal from the dosage detection unit 20. The analog/digital converting unit 40 is provided to convert the amplified determined signal into a digital signal. The microcomputer operation unit 41 is provided to determine the desired dosage from the digital signal based on a predetermined value (dosage control software), and send a signal representing the desired dosage to the thermal acupuncture and moxibustion digital/analog signal amplifier 50. The thermal acupuncture and moxibustion digital/analog signal amplifier 50 converts the received dosage signal into an analog signal and amplify the same. The thermal acupuncture and moxibustion driving unit 60 is connected to the output of the thermal acupuncture and moxibustion digital/analog signal amplifier 50 for driving a heating wire 75 so as to adjust the temperature.

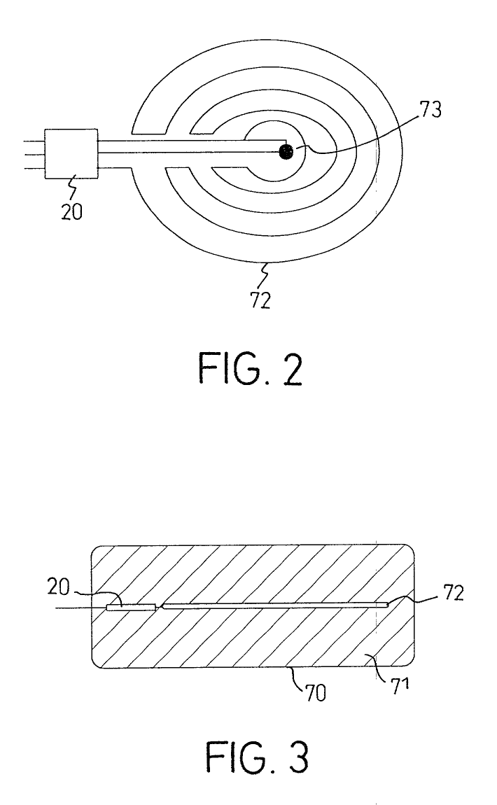

FIG. 2 and FIG. 3 depict the internal structure of a thermal acupuncture and moxibustion unit 70 in accordance with the thermal acupuncture and moxibustion device of the present invention. As shown, there is provided a heating coil circuit 72, the temperature sensor 73, and the dosage detection unit 20 (also with reference to FIGS. 1A and 1B), and insulating heat conductive material 71 encloses the heating coil circuit 72, the temperature sensor 73, and the dosage detection unit 20.

The dosage detection unit 20 comprises an operational amplifier U5A and a variable resistor VR1, wherein the input of the operational amplifier U5A is connected to the output of the temperature sensor 73, and the output of the operational amplifier U5A is connected to the buffering amplifier 30.

The buffering amplifier 30 comprises an operational amplifier U2B and several resistors, wherein the input of the operational amplifier U2B is connected to the output of the operational amplifier U5A of the dosage detection unit 20, and the output of the operational amplifier U2B is connected to the analog/digital converting unit 40.

The analog/digital converting unit 40 comprises an analog/digital converter U3 for converting the analog signal into a digital signal, and send the same to the microcomputer operation unit 41.

The microcomputer operation unit 41 comprises a microprocessor U4 for controlling the desired dosage and sending pulse signals representing the desired dosage to the thermal acupuncture and moxibustion digital/analog signal amplifier 50.

The thermal acupuncture and moxibustion digital/analog signal amplifier 50 comprises operational amplifiers U2D and U2C. The input of the operational amplifier U2C is connected to the output of the microprocessor U4, and the output of the operational amplifier U2C is connected to the input of the operational amplifier U2D. The output of the operational amplifier U2D is connected to the thermal acupuncture and moxibustion driving unit 60.

The thermal acupuncture and moxibustion driving unit 60 comprises a light emitting diode D5, a transistor Q1 and the heating wire 75. The base of the transistor Q1 is connected to the output of the thermal acupuncture and moxibustion digital/analog signal amplifier 50, and the collector of the transistor Q1 is connected to the heating wire 75.

When the heating wire 75 is driven and heated, a thermal acupuncture and moxibustion therapy effect is generated and applied to the human body, such a thermal effect is also sent to the temperature sensor 73 for sensing a temperature signal, and transferring the same to the dosage detection unit 20 for being processed by a comparator composed of the operational amplifier U5A. The dosage detection unit 20 analyzes the signal and sends the analyzed signal to the buffering amplifier 30 for amplifying the same. The amplified signal is sent to the analog/digital converter U3 of the analog/digital converting unit 40 for being converted from an analog signal into a digital signal. The digital signal is sent to the microprocessor U4 of the microcomputer operation unit 41 for being processed by the microprocessor U4. The processed signal is sent to the thermal acupuncture and moxibustion digital/analog signal amplifier 50, and after being processed by two stages of amplifiers U2C and U2D, the pulse signal is sent to the thermal acupuncture and moxibustion driving unit 60. The transistor Q1 drives the heating wire 75 to increase the temperature thereby providing a thermal acupuncture and moxibustion therapy (such an operation is indicated by the light emitting diode).

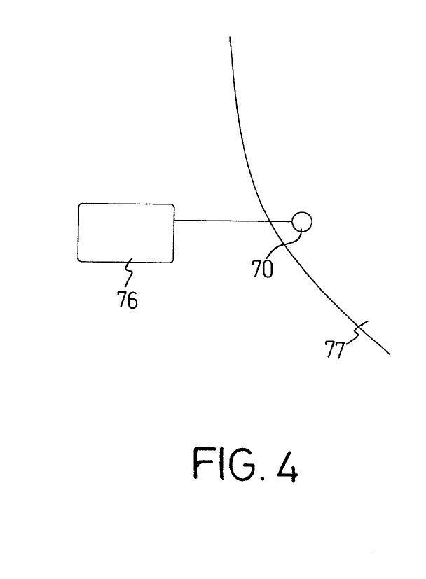

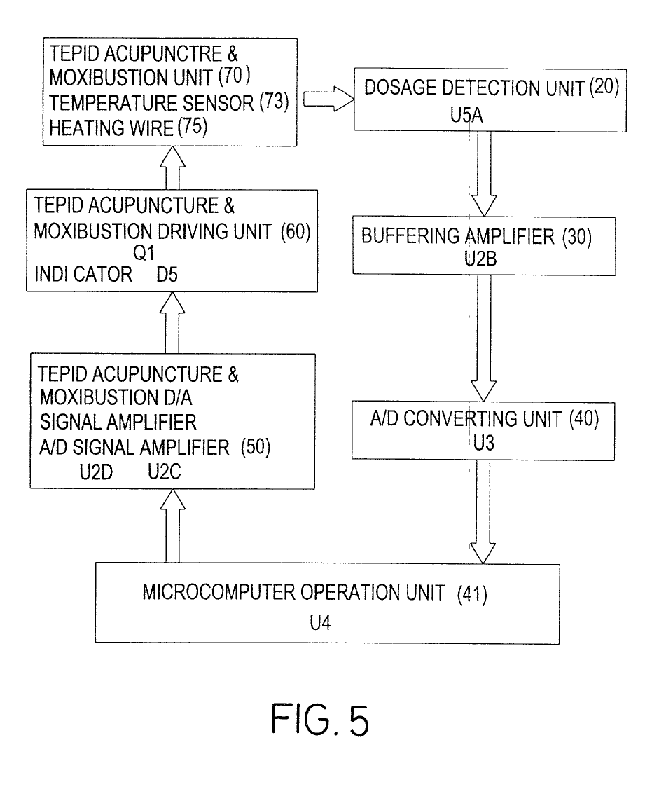

In view of the foregoing, the circuit structure of the thermal acupuncture and moxibustion device in accordance with the present invention is known, and its operational flow is illustrated in FIG. 5. Furthermore, a practical application of the thermal acupuncture and moxibustion device in accordance with the present invention is shown in FIG. 4, wherein the thermal acupuncture and moxibustion unit 70 is attached to the acupuncture point of a human body 77. With the use of the thermal acupuncture and moxibustion temperature control circuit 76, the temperature of the thermal acupuncture and moxibustion unit 70 can be controlled to stimulate the human body, so as to achieve the thermal acupuncture and moxibustion therapy effect.

Although the present invention has been explained in relation to its preferred embodiment, it is to be understood that many other possible modifications and variations can be made without departing from the spirit and scope of the invention as hereinafter claimed.

What is claimed is:

1. A thermal acupuncture and moxibustion device having a thermal acupuncture and moxibustion unit and a temperature control circuit, the temperature control circuit comprising:
a dosage detection unit connected to a temperature sensor for decoding and analyzing signals from the temperature sensor;
a buffering amplifier for amplifying signals from the dosage detection unit;
an analog/digital converting unit for converting the amplified signals into digital signals;
a microcomputer operation unit for determining a desired dosage from the digital signals based on a dosage control software, and sending out pulse signals representing the desired dosage;
a thermal acupuncture and moxibustion digital/analog signal amplifier for converting the pulse signals into analog signals and amplifying the same; and
a thermal acupuncture and moxibustion driving unit connected to an output of the thermal acupuncture and moxibustion digital/analog signal amplifier for driving a heating wire so as to adjust the temperature of the thermal acupuncture and moxibustion unit.

2. The thermal acupuncture and moxibustion device as claimed in claim 1, wherein, in the thermal acupuncture and moxibustion unit, there is provided a heating coil circuit, the temperature sensor, and the dosage detection unit all enclosed by insulating heat conductive material.

3. The thermal acupuncture and moxibustion device as claimed in claim 1, wherein the dosage detection unit comprises an operational amplifier having an input connected to an output of the temperature sensor, and an output connected to an input of the buffering amplifier.

4. The thermal acupuncture and moxibustion device as claimed in claim 1, wherein the buffering amplifier comprises an operational amplifier having an input connected to an output of the dosage detection unit, and an output connected to the analog/digital converting unit.

5. The thermal acupuncture and moxibustion device as claimed in claim 1, wherein the analog/digital converting unit comprises an analog/digital converter for receiving analog signals from the buffering amplifier and converting the same into digital signals for sending to the microcomputer operation unit.

6. The thermal acupuncture and moxibustion device as claimed in claim 1, wherein the microcomputer operation unit comprises a microprocessor and a dosage control software for receiving signals from the analog/digital converting unit to determine a desired dosage and sending out pulse signals representing the desired dosage.

7. The thermal acupuncture and moxibustion device as claimed in claim 1, wherein the thermal acupuncture and moxibustion digital/analog signal amplifier comprises a first operational amplifier and a second operational amplifier, the second operational amplifier having an input connected to the microcomputer operation unit, and an output connected to an input of the first operational amplifier, the first operational amplifier having an output connected to the thermal acupuncture and moxibustion driving unit.

8. The thermal acupuncture and moxibustion device as claimed in claim 1, wherein the thermal acupuncture and moxibustion driving unit comprises a light emitting diode and a transistor having a base connected to an output of the thermal acupuncture and moxibustion digital/analog signal amplifier, and a collector connected to a heating wire.
